(19) United States
(12) Patent Application Publication
(10) Pub. No.: US 2022/0062895 A1
(43) Pub. Date: Mar. 3, 2022

(54) Microfluidic Device And Method Of Assaying For Immune Cell Exhaustion Using Same

(71) Applicant: Wisconsin Alumni Research Foundation, Madison, WI (US)

(72) Inventors: David J. Beebe, Monona, WI (US); Patrick H. McMinn, Madison, WI (US); Jose M. Ayuso, Madison, WI (US); Maria Virumbra!es-Munoz, Madison, WI (US)

(21) Appl. No.: 17/004,290

(22) Filed: Aug. 27, 2020

(51) Int. Cl. B01L 3/00; G01N 33/48

(52) U.S. Cl. CPC B01L 3/502707; G01N 33/48; G01N 2510/00; B01L 2300/0618; B01L 2300/0877; B01L 2300/069

(57) ABSTRACT

A microfluidic device and method of assaying for immune cell exhaustion therewith are provided. The microfluidic device includes a moveable rod positioned across a chamber of a microfluidic device adjacent a first end thereof. Target cells are mixed into a hydrogel and the hydrogel is injected into the chamber about the moveable rod. The hydrogel is polymerized in. the chamber and the moveable rod is removed from the hydrogel so as to form a passageway in the hydrogel. The passageway is filled with a solution including immune cells. The immune cells migrate/diffuse into the hydrogel. A gradient of nutrients is formed in the chamber from. the first end to a second end of the chamber. One or more biopsies of the hydrogel may be taken at user selected location(s) of the chamber.

### FIELD OF THE INVENTION

This invention relates generally to the study of solid tumors, and in particular, to a microfluidic device and a method of assaying for cell exhaustion in a mimicked solid tumor using the same.

### BACKGROUND AND SUMMARY OF THE INVENTION

Solid tumors are highly heterogenous and plastic systems. As solid tumors grow, the accelerated tumor metabolism, combined with an insufficient blood supply to support this uncontrolled metabolism, lead to nutrient exhaustion in the tumor microenvironment. Simultaneously, cellular waste products accumulate in the innermost regions of the tumor. In this context, one of the main waste products is lactic acid, which also causes a pH drop at the core of the tumor.

In view of the foregoing, it can be understood that tumor cells generate an extremely harsh microenvironment characterized by gradients of nutrient exhaustion, waste product accumulation, and pH across the solid tumor mass. Thus, tumor cells located near blood vessels tend to have enough. nutrients to keep growing and form a proliferative outer perimeter. Conversely, those cells located. in the innermost region of the tumor tend to die of nutrient starvation, thereby generating a necrotic core in the center of the tumor. However, those cells located between the proliferative rim and the necrotic core of the tumor play a critical role in tumor development.

In this intermediate layer between the proliferative rim and the necrotic core of a tumor, tumor cells grow in an environment characterized by moderate starvation, hypoxia and acidic pH. However, there are still some nutrients present, as well as, metabolic intermediates that were not consumed by the proliferative cells at the outer perimeter. Under these circumstances, tumor cells in. the intermediate layer adapt their metabolic program to survive within the surrounding harsh microenvironment. Cancer cells decrease or even completely stop their proliferation rate to minimize nutrient consumption, leading to a population of quiescent tumor cells. These quiescent cells activate alternative metabolic pathways and different survival responses (e.g., apoptosis, resistance, starvation-induced DNA. protection). Quiescent cells can. negatively influence patient outcome because quiescent cells evade most chemotherapy agents (e.g., doxorubicin, paclitaxel, and cisplatin), which only target proliferating cells, usually located at the rim of the tumor. As such, these quiescent cells inside the tumor may remain impervious to the treatment.

It has been found that long-term exposure to the chemotherapy drug enables quiescent cells to develop drug resistance mechanisms (e.g., increased drug efflux, blockade of drug uptake proteins, overexpression of detoxifying systems and DNA repair mechanisms or apoptosis evasion). Once the outer proliferative rim is destroyed, these chemotherapy-resistant cells are exposed to high amounts of nutrients, thereby causing cell proliferation to resume, leading to a chemotherapy drug-resistant relapse. In order to find effective therapies capable of targeting these heterogeneous cell populations in the solid tumor, in vitro models are needed to recapitulate the metabolic heterogeneity of the solid tumor microenvironment. In this context, multicellular spheroids represent one of the most traditional 3D in vitro models to study solid tumors. Cancer spheroids exhibit many of the characteristics of solid tumors (e.g., proliferating rim, quiescent region, necrotic core, acidosis, gradients of nutrients). However, to generate these gradients and the necrotic core, the spheroid size must be at least a few hundred microns e.g., approximately 400 microns), making it inaccessible by most microscopy techniques. Another challenge regarding multicellular spheroids is the fact that hypoxia and nutrient gradients appear together, which entangles cellular alterations caused by hypoxia and nutrient starvation. Finally, selectively retrieving the cells from different locations of the spheroid (e.g., proliferating periphery vs. quiescent layer) for downstream. analysis is extremely challenging.

In view of the foregoing, microfluidic devices have become an interesting alternative to more traditional methods to mimic solid tumors. in fact, previous studies have demonstrated the capacity of microfluidic devices to generate gradients of oxygen, nutrients, pH, growth factors and cell viability. However, none of these models enable selective retrieval of cells from different locations in the microdevice, which is essential to decipher the cellular metabolic adaptions under varying microenvironments.

Therefore, it is a primary object and feature of the present invention to provide a microfluidic device for modeling a tumor slice.

It is a further object and feature of the present invention to provide a microfluidic device for modeling a tumor slice wherein nutrient starvation and pH gradients may be mimicked.

It is a further object and feature of the present invention to provide a microfluidic device for modeling a tumor slice which allows for the selective retrieval of cells from the tumor slice for downstream analysis.

It is a still further object and feature of the present invention to provide a method of assaying for immune cell exhaustion in a tumor slice model.

In accordance with the present invention, a microlluidic device with spatially controlled cell isolation capacity is provided. The microfluidic device includes a body having an upper surface and a chamber within the body. The chamber is defined by first and second sides, first and second ends, an upper surface and a lower surface. First and second gradient ports communicate with the chamber. A moveable rod is positionable in the chamber and has a first end supportable by the first gradient port and a second end supportable by the second gradient port.

A first plurality of diffusion ports extends into the upper surface of the body and communicates with the chamber. The first plurality of diffusion ports is axially spaced along an. axis extending though the first and second sides of the chamber. A second plurality of diffusion ports extends into the upper surface of the body and communicates with the chamber. The second plurality of diffusion ports is axially spaced along an axis extending though. the first and second sides of the chamber and parallel to the axis along which the first plurality of diffusion ports is axially spaced.

The microfluidic device may also include a hydrogel polymerized in the chamber. The cells are receivable in the hydrogel. The rod is moveable between a first position wherein the rod is within the hydrogel polymerized in the chamber and a second position wherein the rod is removed from the chamber. The hydrogel defines a tubular passageway extending from the first gradient port and the second gradient port with the rod in the second position. The tubular passageway extends along an axis extending between the first and second sides of the chamber. The axis of the tubular passageway is closer to the first end of the chamber than the second end of the chamber.

The first end of the chamber is defined by a generally arcuate first end wall and the second end of the chamber is defined by a generally arcuate second end wall. A first loading port extends from upper surface to the chamber at a location adjacent the first end of the chamber and a second loading port extends from upper surface to the chamber at a location adjacent the second end of the chamber.

In accordance with a further aspect of the present invention, a method of mimicking a solid tumor within a microfluidic device is provided. The method includes the steps of mixing cells into a hydrogel and injecting the mixture into a chamber of the microfluidic device. The chamber has first and second ends and. first and second sides. A passageway is formed through the mixture in the chamber and filled with a solution. The step of forming the passageway through the mixture includes the steps of positioning a rod in. the chamber in. the microfluidic device; solidifying the mixture within the chamber; and withdrawing the rod from the solidified mixture to form the passageway. The rod is positioned adjacent the first end of the chamber.

Nutrients are allowed to diffuse into the mixture adjacent the first, end of the chamber. A gradient of nutrients is formed in the chamber from the first end to second end. The gradient of nutrients in the chamber causes the cells in the mixture in the chamber to form a first population of proliferating cells adjacent the first end of the chamber, a second population of dead cells adjacent the second end of the chamber, and a third population of stationary cells therebetween. It is contemplated to control an oxygen concentration in the chamber and to take a biopsy of the mixture at a user selected location.

In accordance with a still further aspect of the present invention, a method of assaying for immune cell exhaustion is provided. The method includes the steps of mixing target cells into a hydrogel to form a mixture and positioning a moveable rod across a chamber of a microfluidic device adjacent a first end thereof. The hydrogel is injected into the chamber about the moveable rod and polymerized. The moveable rod is removed from the hydrogel to form a passageway in the hydrogel. The passageway is filled with a solution including immune cells. The immune cells migrate into the hydrogel. A gradient of nutrients is formed in the chamber from the first end to a second end of the chamber. A biopsy of the hydrogel is taken at a user selected location of the chamber.

The chamber includes first and second sides interconnecting the first and second ends. The microfluidic device includes a body defining the chamber and having an upper surface; first and second gradient ports communicating with the passageway; and a first plurality of diffusion ports extending into the upper surface of the body and communicating with the chamber. The first plurality of diffusion ports is axially spaced along an axis extending though the first and second sides of the chamber. A second plurality of diffusion ports extends into the upper surface of the body and. communicates with the chamber. The second plurality of diffusion ports is axially spaced along an axis extending though the first and second sides of the chamber and parallel to the axis along which the first plurality of diffusion ports is axially spaced.

The step of forming the gradient of nutrients in the chamber includes the steps of depositing nutrients on at least one of the first and second plurality of diffusion ports and allowing the nutrient to diffuse into the chamber through the at least one of the first and second plurality of diffusion ports. The step of filling the passageway with the solution includes the step of injecting the solution into the passageway through at least one of the first and second gradient ports. The moveable rod may be removed from the hydrogel by grasping an end of the rod through one of the first and second gradient ports and pulling the rod out of the hydrogel through the one of the first and second gradient ports.

The body includes a first loading port extending from upper surface to the chamber at a location adjacent the first end of the chamber and a second loading port extending from upper surface to the chamber at a location adjacent the second end of the chamber. The hydrogel is injected into the chamber through at least one of the first and second loading ports. The first end of the chamber is defined by a generally arcuate first end wall and the second end of the chamber is defined by a generally arcuate second end wall.

The gradient of nutrients in the chamber causes the cells in the hydrogel to form a first population of proliferating cells adjacent the first end of the chamber, a second population of dead cells adjacent the second end of the chamber, and a third population of stationary cells therebetween. The oxygen concentration in the chamber may be controlled.

### BRIEF DESCRIPTION OF THE DRAWINGS

The drawings furnished herewith illustrate a preferred construction of the present invention in which the above advantages and features are clearly disclosed as well as others which will be readily understood from the following description of the illustrated embodiment.

In the drawings:

FIG. 1 is an exploded, isometric view of a microfluidic device for effectuating a methodology in accordance with the present invention;

FIG. 2 is an isometric view of a microfluidic device for effectuating a methodology in accordance with the present invention;

FIG. 3 is a cross-sectional view of the microfluidic device taken along line 3-3 showing a step for effectuating the methodology of the present invention;

FIG. 3B is an enlarged, cross-sectional view, similar to FIG. 3, wherein the microfluidic device includes a medium reservoir;

FIG. 4 is a cross-sectional view of the microfluidic device, similar to showing a further step for effectuating the methodology of the present invention;

FIG. 5 is a cross-sectional view of the microfluidic device, similar to FIG. 3, showing a still further step for effectuating the methodology of the present invention; and

FIG. 6 is an isometric view of a lower layer of the microfluidic device showing a still further step for effectuating the methodology of the present invention.

### DETAILED DESCRIPTION OF THE DRAWINGS

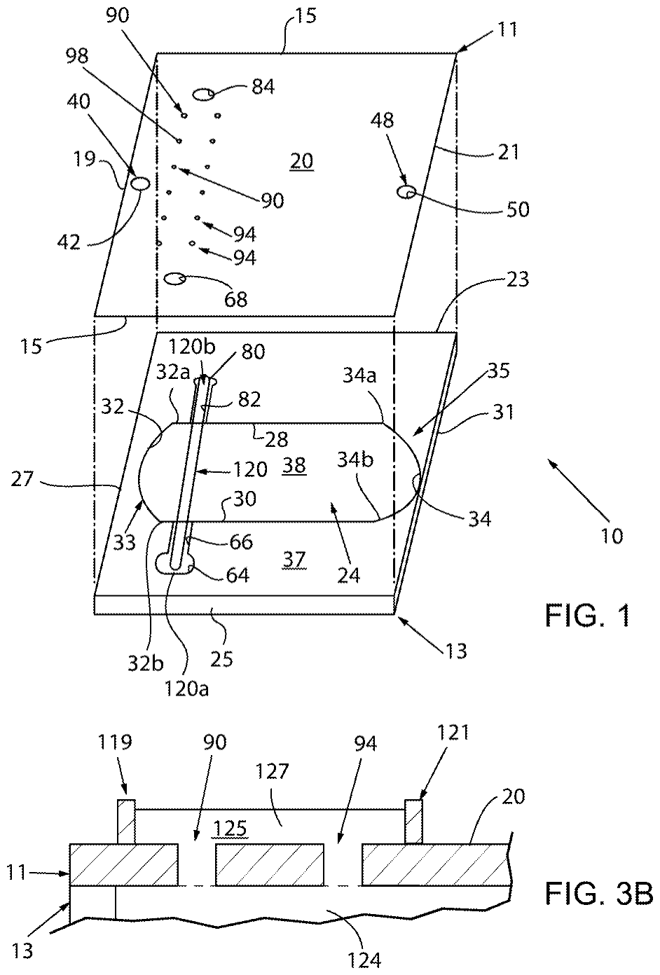

Referring to FIG. 1, a microfluidic device for effectuating the methodology of the present invention is generally designated by the reference numeral 10. It is contemplated to fabricate microfluidic device 10 from an oxygen permeable material, e.g. polydimethylsiloxane (PDMS), for reasons hereinafter described. However, it can be appreciated that microfluidic device 10 may be fabricated from other materials without deviating from the scope of the present invention.

Microfluidic device 10 is fabricated from upper and lower polydimethylsiloxane (PDMS) layers 11 and 13, respectively. Upper layer 11 is defined by first and second generally parallel sides 15 and 17, respectively, interconnected by first and second generally parallel ends 19 and 21, respectively, perpendicular thereto. Upper layer 11 further includes upwardly directed surface 20 and downwardly directed surface 22, FIGS. 3-5.

Lower layer 13 is defined by first and second generally parallel sides 23 and 25, respectively, interconnected by first and second generally parallel ends 27 and 31, respectively, perpendicular thereto. It is contemplated for lower layer 13 to have identical. dimensions as upper layer 11. Lower layer 13 further includes upwardly directed surface 37 and downwardly directed surface 39, FIGS. 3-5. Upwardly directed surface 37 of lower layer 13 further includes chamber 24 formed therein.

Chamber 24 is defined by first and second, generally parallel, sidewalls 28 and 30, respectively, interconnected convex, first end wall 32, adjacent first end 33 of chamber 24, and convex, second end wall 34, adjacent second end 35 of chamber 24. More specifically, first end 32a of first end wall 32 intersects first sidewall 28 and second end 32b of first end wall 32 intersects second sidewall 30. Similarly, first end 34a of second. end wall 34 intersects first sidewall 28 and second end 34b of second end wall 34 intersects second sidewall 30.

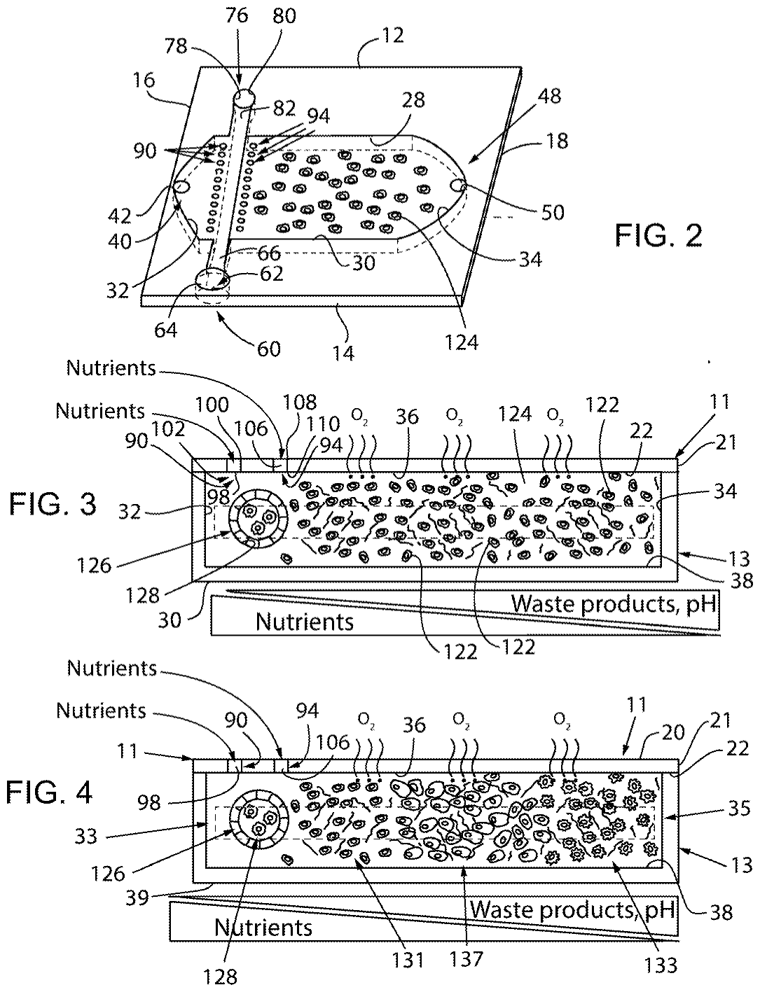

Referring to FIGS. 2-5, to form microfluidic device 10, downwardly directed surface 22 of upper layer 11 is joined to upwardly directed surface 37 of lower layer 13 such that first and second generally parallel sides 15 and 17, respectively, of upper layer 11 are aligned with first and second generally parallel sides 23 and 25, respectively, of lower layer 13 and such that first and second generally parallel ends 19 and 21, respectively, of upper layer 11 are aligned with first and second generally parallel ends 27 and 31, respectively. With upper layer 11 bonded to lower layer 13: first side 15 of upper layer 11 and first side 23 of lower layer 13 define first side 12 of microfluidic device 10; second side 17 of upper layer 11 and second side 25 of lower layer 13 define second side 14 of microfluidic device 10; first end 19 of upper layer 11 and first end 27 of lower layer 13 define first end 16 of microfluidic device 10; and second end 211 of upper layer 11 and second end 31 of lower layer 13 define second end 18 of microfluidic device 10.

Chamber 24 within microfluidic device 10 is further defined by generally parallel upper and lower surfaces 36 and 38, respectively. Upper surface 36 defines a portion of downwardly directed surface 22 of upper layer 11 overlapping chamber 24 with upper and lower layers 11 and 13, respectively, joined together. Upper surface 36 lies in a plane generally parallel to upwardly directed surface 20. Similarly, lower surface 38 partially defining chamber 24 lies in a plane generally parallel to downwardly directed surface 39. As described, channel 24 has a generally elliptical configuration, other configurations are contemplated without deviating from the scope of the present invention.

Microfluidic device 10 further includes first loading port 40 defined by passageway 42 extending through first layer 11 along an axis perpendicular to upwardly directed surface 20. Passageway 42 has a first end communicating with upwardly directed surface 20 and a second end communicating with chamber 24 adjacent to first end wall 32. In addition, microfluidic device 10 further includes second loading port 48 defined by passageway 50 extending through first layer 11 along an axis perpendicular to upwardly directed surface 20. Passageway 50 has a first end communicating with upwardly directed surface 20 and. a second end 54 communicating with chamber 24 adjacent to second end wall 34. As hereinafter described, it is intended for first and second loading ports 40 and 48, respectively, to be used to fill chamber 24 with a media, such as a hydrogel, to provide an environment for cells, e.g., cells 122, as hereinafter described.

Microfluidic device 110 further includes first gradient port 60 defined by passageway 62 having a first. portion 64 extending along an axis generally perpendicular to upwardly directed surface 20 and a second portion 66 extending along an axis generally parallel to upwardly directed surface 20. First portion 64 of passageway 62 has a first end 68 communicating with upwardly directed surface 20 and a second end communicating with a first end of second portion 66 of passageway 62. The second end of second portion 66 of passageway 62 communicates with chamber 24 and intersects second sidewall 30 defining chamber 24.

Second gradient port 76 is defined by passageway 78 having a first portion 80 extending along an axis generally perpendicular to upwardly directed. surface 20 and a second portion 82 extending along an axis generally parallel to upwardly directed surface 20 and coaxial with the axis along which second portion 66 of passageway 62 extends. First portion 80 of passageway 78 has a first end 84 communicating with upwardly directed surface 20 and a second end communicating with a first end of second portion of passageway 78. The second end of second portion 82 of passageway 78 communicates with chamber 24 and intersects first. sidewall 28 defining chamber 24.

Microfluidic device 10 further includes a first set diffusion ports 90 and a second. set diffusion ports 94. Each diffusion. port of the first set of diffusion ports 90 is defined by passageway 98 extending along an axis perpendicular to upwardly directed surface 20. It is contemplated for the axes of passageways 98 to lie in a common plane. Each passageway 98 has a first end 100 communicating with upwardly directed surface 20 and a second end 102 communicating with chamber 24 and intersecting upper surface 36. The diffusion ports of the first set of diffusion ports 90 are axially spaced and lie along a first diffusion port axis such that second ends 102 of passageways 98 of the first set of diffusion ports 90 are spaced between first and second, generally parallel, sidewalls 28 and 30, respectively. It is contemplated for the first diffusion port axis to pass through a location in proximity to the intersection of first end 32a of first end wall 32 with first sidewall 28 and through a location in proximity to the intersection of second end 32b of first end wall 32 with second sidewall 30.

Similarly, each diffusion port of second set of diffusion ports 94 is defined by passageway 106 extending along an axis perpendicular to upwardly directed surface 20 such that the axes of passageways 106 lie in a common plane. Each passageway 106 has a first end 108 communicating with upwardly directed surface 20 and a second end 110 communicating with chamber 24 and intersecting upper surface 36. The diffusion ports of second set of diffusion ports 94 are axially spaced and lie along a second diffusion port axis generally parallel to and spaced from the first diffusion port axis such that second ends 110 of passageways 106 of diffusion ports of the second set of diffusion ports 94 are spaced between first and second, generally parallel, sidewalls 28 and 30, respectively. It is contemplated for each diffusion port of the first set of diffusion ports 92 to be transversely aligned with a corresponding diffusion port of the second set of diffusion ports 94.

Referring back to FIG. 1, in operation, rod 120 may be positioned so as to extend across chamber 24 before first and second layers 11 and 13, respectively, of microfluidic device 10 are joined together. Alternatively, with first and second layers 11 and 13, respectively, of microfluidic device 10 are joined, rod 120 may be inserted through passageway 62 of first gradient port 60, chamber 24 and passageway 78 of second gradient port 76 such that rod 120 extends through chamber 24, is supported at first end 120a of rod 120 within first gradient port 60 and is supported at second end 120b of rod 120 within second gradient port 78. It is contemplated for rod 120 to be fabricated from PDMS. However, rod 120 may be fabricated from other materials without deviating from the scope of the present invention. In the depicted embodiment, rod 120 extends along an axis parallel to and disposed between the plane in which the axes of passageways 98 lie and the plane in which the axes of passageways 106 lie.

To mimic a solid tumor within chamber 24 of microfluidic device 10, selected cells 122 (e.g., cancer cells) are mixed with a media, such as hydrogel 124 or the like, and injected into chamber 24 through passageway 42 of first loading port 40, in any conventional manner. Passageway 50 in second loading port 48 allows for air in chamber 24 to exit chamber 24 during the loading of hydrogel 124 therein. Once hydrogel 124 fills chamber 24, hydrogel 124 is polymerized. For example, hydrogel 124 may be exposed to predetermined stimulus (e.g., heat or light) or maintained at a desired temperature for a desired time period (e.g., at room temperature for a desired number of minutes).

After hydrogel 124 in chamber 24 is polymerized, rod 120 is removed from polymerized hydrogel 124 in chamber 24. By way of example, a pair of sterilized tweezers may be inserted into one of first gradient port 60 and second gradient port 76 to grasp a corresponding end 120a and 120b, respectively, of rod 120 and remove rod 120 from passageway 62 of first gradient port 60, chamber 24 and passageway 78 of second gradient port 76. Referring to FIGS. 3-6, with rod 120 removed from passageway 62 of first gradient port 60, chamber 24 and passageway 78 of second gradient port 76, a lumen model or generally tubular passageway 126 extends through polymerized hydrogel 124 between the second end of second portion 66 of passageway 62 of first gradient port 60 and the second end of second portion 82 of passageway 78 of second gradient port 76. Tubular passageway 126 is defined by tubular surface 128 of polymerized hydrogel 124 and includes a first opening 130 communicating with first gradient port 60 and a second opening 132 communicating with second gradient port 78.

As noted above, PDMS is a gas permeable material, thereby allowing for an oxygen profile across hydrogel 124 in chamber 24, FIGS. 3-4. it can be understood that the oxygen concentration in chamber 24 can be controlled by adjusting the oxygen tension in the surrounding environment. Thus, it is contemplated for microfluidic device 10 to be cultured in an incubator with controlled oxygen tension thereon to allow for a desired oxygen concentrations within chamber 24.

Nutrients may be provided to cells 122 through first and second sets of diffusion ports 90 and 94, respectively. More specifically, in order to ensure the nutrients diffuse homogenously across chamber 24, nutrients may be deposited on upwardly directed surface 20. The nutrients on upwardly directed surface 20 pass through the diffusion ports of first and second sets of diffusion ports 90 and 94, respectively, and diffuse into hydrogel 124 in chamber 24.

Referring to FIG. 3B, by way of example, medium reservoir 119 may be provided on upwardly directed surface 20 of upper layer 11. Medium reservoir 119 is defined by a vertically extending wall. 121 extending from upwardly directed surface 20 of upper layer 1 and about first and second sets diffusion ports 90 and 94, respectively. Inner surface 123 of wall 121 defines cavity 125 for receiving nutrients 127 therein. Cavity 125 communicates with first and second sets diffusion ports 90 and 94, respectively, and allows for nutrients 127 diffuse through first and second sets diffusion ports 90 and 94, respectively, and nourish cells 122 in hydrogel 124 within chamber 24. It can be appreciated that dimensions and configuration of medium reservoir 119 may be varied without deviating from the scope of the present invention.

Over time, after nutrients :127 diffuse through first and second sets diffusion ports 90 and 94, respectively, a gradient of nutrients is formed in hydrogel 124 from first end 33 to second end 35 of chamber 24, FIGS. 3-5. As depicted. in FIG. 4, the gradient of nutrients in chamber 24 generates three different cell populations in chamber 24, namely, proliferating cells 131 adjacent first end 33 of chamber 24 and tubular passageway 126, dead cells 133 adjacent second end 35 of chamber 24, and stationary cells 137 therebetween. Further, it can be understood that cellular waste products within hydrogel 124 accumulate adjacent second end 35 of chamber 24, thereby causing a corresponding pH drop. As such, it can be understood that cells 122 in chamber 24 adjacent. tubular passageway 126 mimic cells at the outermost regions of a tumor and cells 122 at second end 35 of chamber 24 mimic those cells at the innermost regions of the tumor. As such, it can be appreciated that the cells 122 in hydrogel 124 in chamber 24 mimic a solid tumor.

Thereafter, in order to study the effects of a desired media, cells, cytokines, etc., e.g., a solution 135 including natural killer cells 136 (also known as NK cells, K cells, and killer cells), on a solid tumor, it is contemplated to deposited solution 135 in first gradient port 60 so as to flow though tabular passageway 126 into second gradient port 76, FIG. 5. NK cells 136 in tubular passageway 126 migrate/diffuse through tubular surface 128 into hydrogel 124 in chamber 24 thereby forming a gradient of NK cells 136 within hydrogel 124 from tubular passageway 126 to second end 35 of chamber 24.

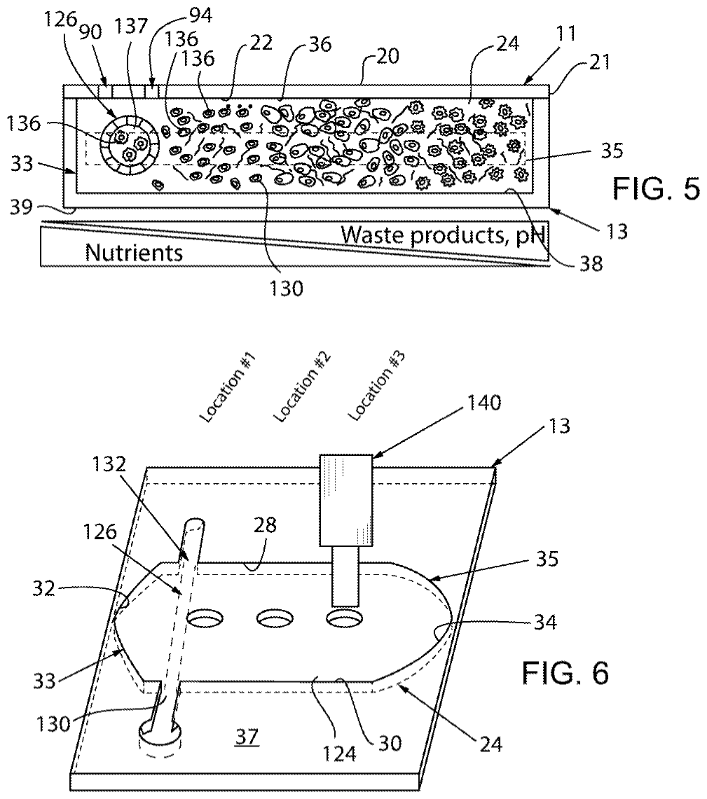

Referring to FIG. 6, in order to selectively retrieve cells 122 from microfluidic device 10 to ascertain the effects of solution 135 thereon, it is contemplated to remove upper layer 11 from microfluidic device 10 to expose hydrogel 124 in chamber 24. Using biopsy punch 140, one or more hydrogel punches may be obtained at the different locations, e.g., location #1, location #2, and location#3, spaced from tubular passageway 126 by selected distances. Cells 122 may be removed from the hydrogel punches in any conventional manner to allow for further downstream processing.

It can be appreciated that the structure of microfluidic device may be modified to facilitate the study of solid tumors. By way of example, it is contemplated to provide one or more additional tubular passageways through hydrogel 124. More specifically, microfluidic device 10 may include one or more additional pairs of gradient ports similar to first and second gradient ports 60 and 76) at desired locations. A user may provide a rod passing though chamber 24 at a desired location and having a first end supported within one of the addition pair of gradient ports and a second end supported within. the other of the additional pair of gradient ports prior to loading chamber 24 with the hydrogel. After the hydrogel is polymerized, the rod may be removed to create an additional tubular passageway through hydrogel 124. An alternate solution may be deposited in the additional tubular passageway and allowed to migrate/diffuse into hydrogel 124 in chamber 24. Further, it can be appreciated that additional diffusion ports may be provided at different location of microfludic device 10 to allow for additional or alternate media to be provided to cells 24 in hydrogel 124.

Various modes of carrying out the invention are contemplated as being within the scope of the following claims particularly pointing out and distinctly claiming the subject matter that is regarded as the invention.

We claim:

1. A microfluidic device with spatially controlled cell isolation capacity, comprising:
a body having:
an upper surface;
a chamber within the body, the chamber defined by first and second sides, first and second ends, an upper surface and a lower surface; and
first and second gradient ports communicating with the chamber;
a moveable rod positionable in the chamber and having a first end supportable by the first gradient port and a second end supportable by the second gradient port.

2. The microfluidic device of claim I further comprising a first plurality of diffusion ports extending into the upper surface of the body and communicating with the chamber, the first plurality of diffusion ports axially spaced along an axis extending though the first and second sides of the chamber.

3. The microfluidic device of claim I further comprising a. second plurality of diffusion ports extending into the upper surface of the body and communicating with the chamber, the second plurality of diffusion ports axially spaced along an axis extending though the first and second sides of the chamber and parallel to the axis along which the first plurality of diffusion ports is axially spaced.

4. The microfluidic device of claim 1 further comprising a hydrogel polymerized in the chamber, the cells being receivable in the hydrogel.

5. The microfluidic device of claim 4 wherein the rod is moveable between a first position wherein the rod is within the hydrogel polymerized in the chamber and a second position wherein the rod is removed from the chamber.

6. The microfluidic device of claim 5 wherein the hydrogel defines a tubular passageway extending from the first gradient port and the second gradient port with the rod in the second position.

7. The microfluidic device of claim 6 wherein tubular passageway extends along an axis extending between the first and second sides of the chamber, the axis of the tubular passageway being closer to the first end of the chamber than the second end of the chamber.

8. The microfluidic device of claim 1 wherein the first end of the chamber is defined by a generally arcuate first end wall.

9. The microfluidic device of claim 8 wherein the second end of the chamber is defined by a generally arcuate second end wall.

10. The microfluidic device of claim 1 further comprising a first loading port extending from upper surface to the chamber at a location adjacent the first end of the chamber and a second loading port extending from upper surface to the chamber at a location adjacent the second end of the chamber.

11. A method of mimicking a solid tumor within a microfluidic device, comprising the steps of:
mixing cells into a hydrogel;
injecting the mixture into a chamber of the microfluidic device, the chamber device having first and second ends and first and second sides;
forming a passageway through the mixture in the chamber; and
filling the passageway with a solution.

12. The method of claim 11 wherein the step of forming the passageway through the mixture includes the steps
positioning a rod in the chamber in the microfluidic device;
solidifying the mixture within the chamber; and
withdrawing the rod from the solidified mixture to form the passageway.

13. The method of claim 12 wherein the rod is positioned adjacent first end of the chamber.

14. The method of claim 11 comprising the additional step of allowing nutrients to diffuse into mixture adjacent the first end of the chamber.

15. The method of claim 11 comprising the additional step of forming a gradient of nutrients in the chamber from the first end to second end.

16. The method of claim 15 wherein the gradient of nutrients in the chamber causes the cells in. the mixture in the chamber to form a first population of proliferating cells adjacent the first end of the chamber, a second population of dead cells adjacent the second end of the chamber, and a third population of stationary cells therebetween.

17. The method of claim 11 comprising the additional step of controlling an oxygen concentration in the chamber.

18. The method of claim 11 comprising the additional step of taking a biopsy of the mixture at a user selected location.

19. A method of assaying for immune cell exhaustion, comprising the steps of:
mixing target cells into a hydrogel;
positioning a moveable rod across a chamber of a microfluidic device adjacent a first end thereof;
injecting the hydrogel into the chamber about the moveable rod;
polymerizing the hydrogel in the chamber;
removing the moveable rod from the hydrogel to form a passageway in the hydrogel;
filling the passageway with a solution including immune cells, the immune cells migrating into the hydrogel;
forming a gradient of nutrients in the chamber from the first end to a second end of the chamber; and
taking a biopsy of the hydrogel at a user selected location of the chamber.

20. The method of claim 19 wherein the chamber includes first and second sides interconnecting the first and second ends and the microfluidic device includes:
a body defining the chamber and having:
an upper surface;
first and second gradient ports communicating with the passageway;
a first plurality of diffusion ports extending into the upper surface of the body and communicating with the chamber, the first plurality of diffusion ports axially spaced along an axis extending though the first and second sides of the chamber; and
a second plurality of diffusion ports extending into the upper surface of the body and communicating with the chamber, the second plurality of diffusion ports axially spaced along an axis extending though the first and second sides of the chamber and parallel to the axis along which the first plurality of diffusion ports is axially spaced.

21. The method of claim 20 wherein the step of forming the gradient of nutrients in the chamber includes the steps of depositing; nutrients on at least one of the first and second plurality of diffusion ports and allowing the nutrient to diffuse into the chamber through the at least one of the first and. second plurality of diffusion ports.

22. The method of claim 20 wherein the step of fillip the passageway with the solution includes the step of injecting the solution into the passageway through at least one of the first and second gradient ports.

23. The method of claim 20 wherein the step of removing the moveable rod from the hydrogel includes grasping an end of the rod through one of the first and second gradient ports and pulling the rod out of the hydrogel through the one of the first and second gradient ports.

24. The method of claim 20 wherein the body includes a first loading port extending from upper surface to the chamber at a location adjacent the first end of the chamber and a second loading port extending from upper surface to the chamber at a location adjacent the second end of the chamber and wherein the hydrogel is injected into the chamber through at least one of the first and second loading ports.

25. The method of claim 19 wherein the fast end of the chamber is defined by a generally arcuate first end wall.

26. The method of claim 19 wherein the second end of the chamber is defined by a generally arcuate second end wall.

27. The method of claim 19 wherein the gradient of nutrients in the chamber causes the cells in the hydrogel to form a first population of proliferating cells adjacent the first end of the chamber, a second population of dead cells adjacent the second end of the chamber, and a third population of stationary cells therebetween.

28. The method of claim 19 comprising the additional step of controlling an oxygen concentration in the chamber.
